(19) United States
(12) Patent Application Publication
(10) Pub. No.: US 2008/0092625 A1
(43) Pub. Date: Apr. 24, 2008

(54) Gas Leak Detector Having An Integral Data Logger

(76) Inventor: Michele Hinnrichs, Solvang, CA (US)

Correspondence Address: QUINN LAW GROUP, PLLC, 39555 ORCHARD HILL PLACE, SUITE # 520, NOVI, MI, 48375, US

(21) Appl. No.: 11/572,273

(22) PCT Filed: Jul. 27, 2004
(86) PCT No.: PCT/US04/24264

§ 371 Date: Jan. 23, 2007

(51) Int. Cl. G01N 33/00

(52) U.S. Cl. 73/23.2

(57) ABSTRACT

A remote sensing device and method operable for detecting and analyzing gases, vapors and flame plumes using imaging. The gas imaging instrument includes a gas-leak imaging device, for example, an Image Multispectral Sensing (IMSS) device (10), enhanced by advanced image processing techniques and micro-miniature circuitry, and includes a GPS (21), a clock and computer means (24) that are collectively operable for logging positional, temporal and gas-leak data. These enhancements provide a portable instrument with the capability to not only remotely detect and image gases, including gas leaks, but additionally provide a record of the position and time the spectrometric gas-leak data were collected in a single device (camera) (25).

### BACKGROUND OF THE INVENTION

1. Field of the Invention

This invention is a gas leak imaging system useful for measuring the gases emanating from a gas leak at a remote location, and, more particularly, provides a method and apparatus for locating gas leaks and recording the geographic coordinates of the leak and the time that the gas leak was detected.

2. Description of the Prior Art

Gas producers and distributors lose millions of dollars annually due to gas leakage from a distribution line or containment facility. A portable instrument with the capability to remotely detect and image gases, including gas leaks, is needed. Such a device and method for finding and identifying gas leaks should preferably further provide an analysis of the gas including chemical species and concentrations.

Image multispectral sensing (IMSS) records the spectrum of individual luminous objects (targets) within an image or scene. IMSS is capable of simultaneously recording the spectrum of light emerging from many different discrete light sources contained within a single field of view. U.S. Pat. No. 5,479,258 to Hinnrichs et al., the contents of which is incorporated herein by reference thereto, discloses an image multispectral sensing device, which provides good spectral resolution for images comprising luminous point objects which have good contrast ratios with respect to the background. Accordingly, the IMSS technology is an example of a gas-leak imaging device that may be employed for detecting and measuring gas leaks.

The IMSS (“Sherlock”) camera, disclosed in U.S. Pat. No. 6,680,778 to the present inventor, is currently marketed as an alternate work practice to Method 21 for the Leak Detection and Repair Program (LDAR), a congressionally mandated program. The current Method 21 is very specific about how the leaks are to be monitored and repaired. This method has been in practice since the early 1990's. It requires considerable data to be logged such as the position (eg: geographic coordinates) of each gas leak, the dates that they are checked and the status of repair, if any. With the new alternate work practice to Method 21, which will allow optical imaging techniques, this same data needs to be logged and tracked as before and to have the ability to keep video image data as part of the data set.

As mentioned above, the ability of IMSS spectrometers to distinguish between an object and a background have been extended to the detection of a non-homogeneous distribution of gases in a volume of gas as described in the above-referenced '778 patent. Although an IMSS device such as the Sherlock camera is an example of a device that is useful for detecting and measuring gas leaks, there remains a need for a gas imaging device which is portable and operable for detecting gas leaks at remote locations, and wherein the apparatus further includes data logging means integral therewith, the data logging means being operable for recording the temporal, positional and other data for each gas-leak measurement (image) in accordance with the requirements of Method 21.

### SUMMARY OF THE INVENTION

The present invention is directed to a gas leak detector that substantially obviate one or more of the limitations of the related art. To achieve these and other advantages and in accordance (with the purpose of the invention, as embodied and broadly described herein, the invention includes a gas imaging device operable for detecting the presence of a gas leak from a gas containment device and generate an image corresponding thereto, and for recording at least the date, time and geographic coordinates corresponding to the image.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a schematic diagram of an exemplary gas-leak detection device comprising an IMSS gas detection instrument in accordance with the prior art, operable for detecting gas leaks.

FIG. 2 is a schematic diagram of a gas detection instrument in accordance with the present invention, operable for detecting and measuring gas leaks and for logging a data set corresponding to each such gas leak measurement.

### DESCRIPTION OF THE PREFERRED EMBODIMENTS

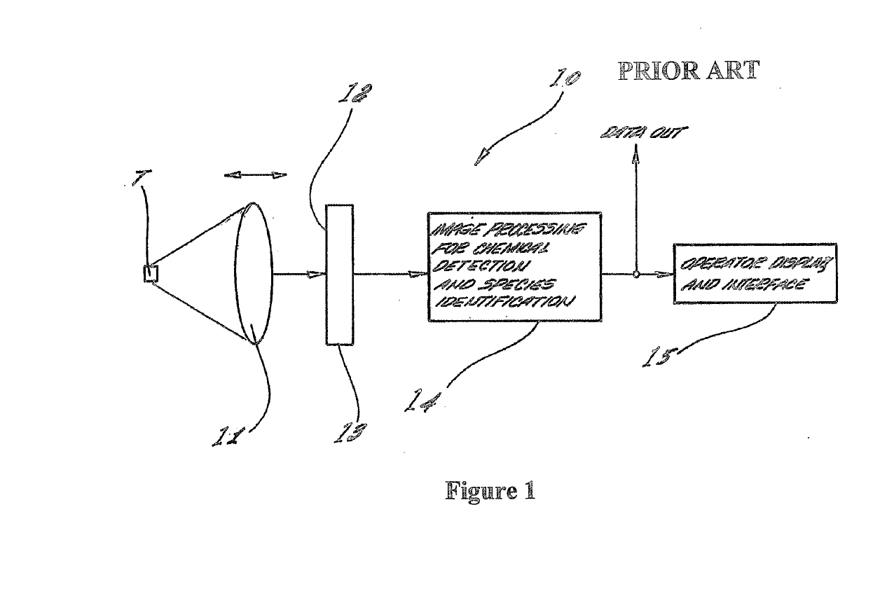

The present invention provides an apparatus and method for identifying the presence of a gas leak at a location remote from the apparatus. It will be understood by an artisan skilled in art of gas-leak detection that while the present invention is described by using an IMSS device as an exemplary gas-leak detector, any gas-leak detection or imaging device may be incorporated with a data logging system to provide a single apparatus operable for performing the function of the present invention. An example of a gas-leak imaging apparatus in accordance with the prior art is an IMSS gas leak imaging device, shown in schematic view at numeral 10 in FIG. 1. The apparatus 10 comprises a diffractive optical element 11 which is used to focus an image of the target chemical T under analysis on a photosensitive surface 12 of a detector 13 (such as a focal plane array (FPA)). An image of the scene under view is formed on the detector 13. The IMSS's diffractive optical element 11 focuses different wavelengths of light at different distances or focal lengths. The distance between the diffractive optical element 11 and the detector 13 is changed to form a series of very narrowband spectral images. The images are stored in the image processing electronics 14. The image processing electronics 14 uses these spectral images to detect and image the target chemical by techniques such as, for example, by comparing the image at the absorption band of the target chemical to images outside the absorption band, using motion detection algorithms, and applying techniques such as principle components analysis. A raw image and a processed image of the target chemical T can be provided to the operator via operator display 15.

The instrument 10 can also be employed to identify an unknown chemical in a volume of gas. In this application, the spectrum of an unknown target chemical T obtained by the instrument is compared with a spectral database of chemical spectra stored in the image processing electronics to identify the chemical species of the target chemical. Concentrations of the target chemical are obtained by determining the absorption (or emission in the case of a flame or plume) of the target chemical at its absorption wavelength (or emission wavelength) compared to images outside the absorption or emission region. While the IMSS “camera” in accordance with the prior art is operable for measuring gas in a volume of space around a container such as a pipe, it lacks means for inputting positional and other data related to the volume of space in which the measurement was made. Accordingly, the image-associated data must be recorded separately and correlated with the image(s) as a separate procedure.

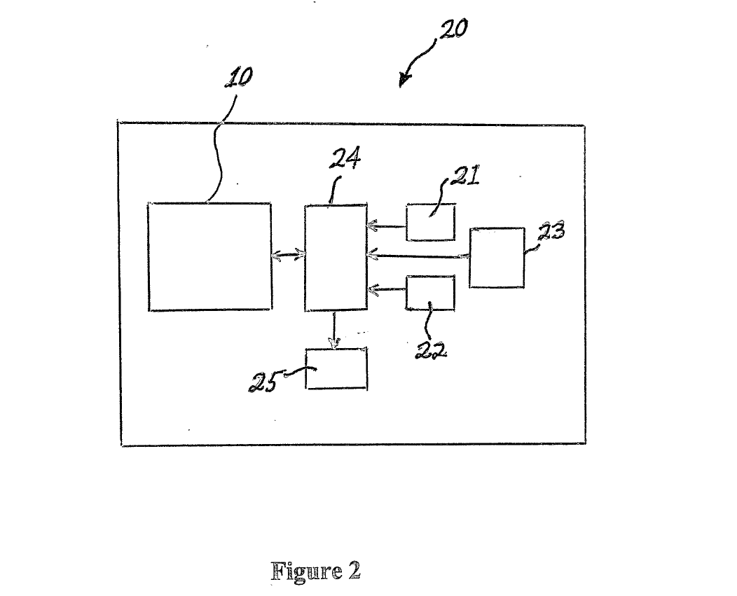

The present invention is directed to a device that substantially obviates one or more of the limitations of the art. To achieve these and other advantages and in accordance with the purpose of the invention, as embodied and broadly described herein, the invention is a device incorporating a gas imaging device and a data logger in a single instrument. FIG. 2 is a schematic diagram of a gas detection instrument 20 in accordance with the present invention, operable for detecting and measuring gas leaks, and for generating an image corresponding to each gas leak measurement and for generating and recording (i.e., “logging”) a data set corresponding to each measurement image.

With continued reference to FIG. 2, the device 20 includes a gas-leak imaging device such as, for example, an IMSS device 10 operable for detecting and measuring a gas leak. The “data out” port (FIG. 1) from the IMSS 10 provides image input to computer means 24. When the image corresponding to a particular gas leak measurement is received by computer means 24, the GPS coordinates of the instrument from a GPS indicating device 21 is logged into the computer means 24, and a clock 22 provides the date and time that the image was generated. A keypad 23 may be employed by the operator to input additional data to the computer means 24 for each image as is required by the particular application. The image and image-associated data set is recorded in a digital recording device 25

In view of the foregoing, and in light of the objectives of the invention, it will be apparent to those skilled in the art that the subject matter of this invention is capable of variation in its detail, and should not be construed to be limited to the specific embodiment of the apparatus which was selected for the purpose of explaining the invention. For example, it will be apparent to the skilled artisan that although the present invention was described employing an IMSS device as the gas leak imaging device, the present invention may include any gas imaging device that includes computer and data logging means housed within a single instrument. The foregoing has been merely a description of one embodiment of the imaging spectrophotometer that may be used to detect gas leaks. Other devices operable for detecting and measuring a gas leak may also be used. The novel feature of the present invention is the inclusion of data logging means within the gas imaging device such that a single instrument may be used to provide an image of a gas leak and generate and log a data set corresponding to each image. The scope of the invention can be determined by reference to the claims appended hereto.

What I claim is:

1. A device operable for detecting the presence of a particular gas in a volume of gas and generating and storing an image corresponding to the presence of the gas in the volume of gas and operable for recording at least the position that the image was generated, the device comprising: (a) a gas detection device operable for visualizing a target gas present in a volume of gas and generating an image thereof, (b) computer means operable for receiving said image; (c) a global position indicator operable for presenting a set of geographical coordinates of said volume of gas to said computer means.

2. The device of claim 1 further comprising a clock operable for presenting date and time said image was generated to said computer means.

3. The device of claim 2 further comprising recording means operable for receiving said image, said coordinates and said time and date from said computer means and providing a permanent record of said image, coordinates and time and date.

4. A device operable for detecting the presence of a particular gas in a volume of gas and for measuring the concentration of the gas and generating and storing an image corresponding to the concentration of the gas in the volume of gas and operable for recording at least the time and position that the image was generated, the device comprising: (a) an IMSS device operable for measuring the concentration of a target gas present in a volume of gas and generating an image thereof, (b) computer means operable for receiving said image; (c) a global position indicator operable for presenting a set of geographical coordinates to said computer means corresponding the the location of the volume of gas; and (d) a clock operable for presenting date and time data to said computer means; and (e) recording means operable for receiving said image, said coordinates and said time and date from said computer means and providing a permanent record of said image, coordinates and time and date.
